(19) United States
(12) Patent Application Publication
(10) Pub. No.: US 2024/0076749 A1
(43) Pub. Date: Mar. 7, 2024

(54) Role of PVT1 in the Diagnosis and Treatment of MYC-Driven Cancer

(71) Applicant: Sanford Burnham Prebys Medical Discovery Institute, La Jolla, CA (US)

(72) Inventors: Anindya BAGCHI, San Diego, CA (US); Ashutosh TIWARI, San Diego, CA (US)

(21) Appl. No.: 18/503,865

(22) Filed: Nov. 7, 2023

Related U.S. Application Data
(62) Division of application No. 17/053,328, filed on Nov. 5, 2020, now Pat. No. 11,866,788.
(60) Provisional application No. 62/668,638, filed on May 8, 2018.

(51) Int. Cl. C12Q 1/6886

(52) U.S. Cl. CPC C12Q 1/6886; C12Q 2600/106; C12Q 2600/156

(57) ABSTRACT

Disclosed herein are methods of diagnosing and treating MYC-driven cancers by detecting a PVT1 splice variant in a biological sample from a subject.

### CROSS-REFERENCE TO RELATED APPLICATIONS

Any and all priority claims identified in the Application Data sheet, or any correction thereto, are hereby incorporated by reference under 37 CFR 1.57. For example, this application is a divisional application of U.S. patent application Ser. No. 17/053,328, filed Nov. 5, 2020, which is a U.S. national stage application of International Patent Application No. PCT/US2019/031349, filed May 8, 2019, which claims the benefit of U.S. Provisional Application No. 62/668,638, filed May 8, 2018, all of which are incorporated herein by reference in their entirety.

### STATEMENT AS TO FEDERALLY SPONSORED RESEARCH

This invention was made with government support under W81XWH-17-1-0461 awarded by the Medical Research and Development Command, and R01 CA200643 awarded by the National Institutes of Health. The government has certain rights in the invention.

### SEQUENCE LISTING

The instant application contains a Sequence Listing which has been submitted in XML file format and is hereby incorporated by reference in its entirety. Said XML copy, created on Oct. 26, 2023, is named 42256-733_401_SL.XML and is 649,294 bytes in size.

### INCORPORATION BY REFERENCE

All publications, patents, and patent applications mentioned in this specification are herein incorporated by reference to the same extent as if each individual publication, patent, or patent application was specifically and individually indicated to be incorporated by reference, and as if set forth in their entireties.

### BACKGROUND OF THE DISCLOSURE

There is a need to for improved diagnosis and therapeutic targeting of MYC-driven cancers.

### SUMMARY OF THE DISCLOSURE

The instant disclosure is based on the observation that PVT1 splice variants are related to c-MYC driven cancers, and further in-depth characterization of the PVT1 splice variants revealed insightful components for therapeutic and diagnostic use.

One embodiment provides a method of identifying a subject as having a MYC-driven cancer, comprising: detecting a presence of a PVT1 splice variant or a peptide encoded by the PVT1 splice variant (PEP) in a biological sample isolated from said subject, and identifying said subject as having a MYC-driven cancer based on the presence of the PVT1 splice variant or the PEP, wherein the PVT1 splice variant comprises PVT1_212. In some embodiments, the method further comprises measuring the expression level of a circular PVT1_212 RNA, PVT1_212 splice variant, or PEP, or both, in the biological sample. In some embodiments, the method further comprises measuring the expression level of c-Myc or MYC, or both, in the biological sample.

In some embodiments, the PEP is a PEPc.

In some embodiments, the PEPc comprises 104 amino acids. In some embodiments, the method comprises measuring the expression level of the PEPc in the biological sample. In some embodiments, the biological sample is a liquid sample. In some embodiments, the liquid sample comprises blood or plasma. In some embodiments, the PEPc comprises a liquid biopsy biomarker for identifying if said subject has a MYC-driven cancer.

In some embodiments, the PEP comprises PEP_{L}. In some embodiments, PEP_{L} comprises 149 amino acids. In some embodiments, the presence or expression levels of the PEP are measured using an antibody against PEPc, an antibody against PEP_{L}, or both.

In some embodiments, the method comprises identifying said subject as having a MYC-driven 8q24 gain cancer. This is determined, in some cases, by the presence of the PVT1_212 splice variant or increased expression level of the PVT_212 splice variant, or the PEP (PEPc or PEP_{L}) relative to reference values. In some embodiments, the method comprises identifying said subject as having a MYC-driven 8q24 gain medulloblastoma, based on an expression level of the PVT1_212 splice variant in the biological sample, wherein the biological sample is a tumor sample isolated from said subject.

In some embodiments, the method further comprises stratifying said subject as a subgroup 3, subgroup 4, Wnt, or Shh type medulloblastoma, based on the expression level of the PVT1_212 splice variant in the tumor sample.

In some embodiments, said subject is stratified as having a subgroup 3 type medulloblastoma if PVT1_212 splice variant expression level is 200 to 1000 fold elevated compared to a reference value. In some embodiments, said subject is stratified as having a subgroup 3, Wnt, or Shh type medulloblastoma if PVT1_212 splice variant expression level is 15 to 200 fold elevated compared to a reference value.

In some embodiments, said subject is stratified as having a subgroup 4 type medulloblastoma if PVT1_212 splice variant expression level is 0 to 15 fold elevated compared to a reference value.

In some embodiments, said subject is identified as having a MYC-driven 8q24 gain medulloblastoma if PVT1_212 splice variant expression level is 200 to 1000 fold elevated compared to a reference value. In some embodiments, the method comprises identifying said subject as having a MYC-driven 8q24 gain cancer, based on an expression level of the PVT1_212 splice variant in the biological sample, wherein the biological sample comprises a tumor sample isolated from said subject. In some embodiments, said subject is identified as having a MYC-driven 8q24 gain cancer if the expression level of the PVT1_212 splice variant is 200 to 1000 fold elevated compared to a reference value. In some embodiments, the reference value comprises expression level of the PVT1_212 splice variant in a biological sample isolated from a subject who does not have a cancer. In some embodiments, the biological sample comprises a tumor sample isolated from said subject and wherein the reference value comprises expression level of the PVT1_212 splice variant in a non-tumor sample from said subject.

One embodiment provides a method of treating cancer in a subject, comprising administering an agent that inhibits a PVT1 splice variant or a peptide encoded by the PVT1 splice variant (PEP), wherein said subject has been identified as having a MYC-driven cancer according to the method of any one of above embodiments.

One embodiment provides a method for selecting a therapy for treating a subject who has a cancer characterized by gain of c-myc, the method comprising (i) detecting a presence of a PVT1 splice variant or a peptide encoded by the PVT1 splice variant (PEP) in a biological sample isolated from said subject, wherein the PVT1 splice variant comprises PVT1_212; and (ii) selecting a therapy comprising an agent that inhibits the PEP for treating said cancer in said subject, if the PVT1 splice variant or the PEP is detected in step (i). In some embodiments, said subject has previously identified as having a cancer characterized by a co-gain of PVT1 and c-Myc. In some embodiments, the co-gain is identified by assaying the copy numbers of PVT1 and c-Myc in the same or a distinct biological sample isolated from said subject and comparing the copy numbers to reference values. In some embodiments, the reference values are copy numbers of PVT1 and c-Myc in a biological sample isolated from a subject who does not have a cancer. In some embodiments, the same or distinct biological sample comprises a tumor sample isolated from said subject and wherein the reference values are copy numbers of PVT1 and c-Myc in a non-tumor sample isolated from said subject. In some embodiments, the co-gain is identified by assaying the copy numbers of PVT1 and c-Myc in the same or a distinct biological sample and comparing with copy numbers of said genes available from the TCGA or ENSEMBL database. In some embodiments, step (i) comprises detecting the presence of the PEP using an anti-PEP antibody, a chromosomal probe specific for the 8q24 locus, or a combination of both. In some embodiments, step (i) comprises measuring the expression level of c-Myc in the biological sample before and after the biological sample is treated with an agent that is specific for exon 3 of the PVT1_212 splice variant, and wherein a reduced expression of c-Myc after treatment of the sample with the agent is indicative of the presence of the PVT1_212 splice variant. In some embodiments, the cancer comprises a 8q24.21 gain cancer. In some embodiments, the cancer comprises medulloblastoma, breast cancer, ovarian cancer, lung cancer, prostate cancer, or a colorectal cancer. In some embodiments, the same or distinct biological sample comprises a biological fluid sample. In some embodiments, the same or distinct biological sample comprises a tumor sample. In some embodiments, the tumor sample comprises a tissue biopsy or a resection.

One embodiment provides a method for characterizing a cancer in a subject, the method comprising: determining a gene expression level of a PVT1 splice variant PVT1_212 in a biological sample isolated from said subject, and characterizing the cancer as a MYC-driven cancer if expression levels of the PVT1_212 is higher than a reference value.

One embodiment provides a method for characterizing a cancer in a subject, the method comprising: detecting a presence of a PVT1 splice variant PVT1_212 in a biological sample isolated from said subject, and characterizing the cancer as a MYC-driven cancer if the PVT1 splice variant is detected in the biological sample.

One embodiment provides a method of treating a cancer in a subject, the method comprising: detecting a presence of a PVT1 splice variant PVT1_212 in a biological sample isolated from said subject, and administering a therapy targeting a peptide encoded by the PVT1_212, wherein said peptide comprises PEPc or PEP_{L}. In some embodiments, said subject has previously identified as having a cancer characterized by a co-gain of PVT1 and c-Myc. In some embodiments, the co-gain is identified by assaying copy numbers of PVT1 and c-Myc in the same or a distinct biological sample isolated from said subject and comparing the copy numbers to reference values. In some embodiments, the reference values are the copy numbers of PVT1 and c-Myc in a biological sample isolated from a subject who does not have a cancer. In some embodiments, the reference values are the copy numbers of PVT1 and c-Myc in a non-tumor sample isolated from said subject. In some embodiments, the co-gain is identified by assaying the copy numbers of PVT1 and c-Myc in the same or a distinct tumor sample isolated from said subject and comparing with copy numbers of said genes available from the TCGA or ENSEMBL database. In some embodiments, the cancer comprises a 8q24.21 gain cancer. In some embodiments, the cancer comprises medulloblastoma, breast cancer, ovarian cancer, lung cancer, prostate cancer, or a colorectal cancer.

One embodiment provides a method of treating a cancer in a subject, the method comprising administering a therapy targeting a peptide encoded by a PVT1 splice variant PVT1_212, wherein the subject has previously been identified as having a cancer characterized by co-gain of PVT1 and c-Myc, wherein said peptide comprises PEPc or PEP_{L}. In some embodiments, the co-gain is identified by assaying copy numbers of PVT1 and c-Myc in a biological sample isolated from said subject and comparing the copy numbers to reference values. In some embodiments, the reference values are the copy numbers of PVT1 and c-Myc in a biological sample isolated from a subject who does not have a cancer. In some embodiments, the reference values are the copy numbers of PVT1 and c-Myc in a non-tumor sample isolated from said subject. In some embodiments, the co-gain is identified by assaying copy numbers of PVT1 and c-Myc in a tumor sample isolated from said subject and comparing with copy numbers of said genes available from the TCGA or ENSEMBL database. In some embodiments, the cancer comprises a 8q24.21 gain cancer. In some embodiments, the cancer comprises medulloblastoma, breast cancer, ovarian cancer, lung cancer, prostate cancer, or a colorectal cancer.

### DETAILED DESCRIPTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Certain Definitions

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” should be assumed to mean an acceptable error range for the particular value.

The terms “individual,” “patient,” or “subject” are used interchangeably. None of the terms require or are limited to situation characterized by the supervision (e.g. constant or intermittent) of a health care worker (e.g. a doctor, a registered nurse, a nurse practitioner, a physician's assistant, an orderly, or a hospice worker).

The term “gene,” as used herein, refers to a segment of nucleic acid that encodes an individual protein or RNA (also referred to as a “coding sequence” or “coding region”), optionally together with associated regulatory regions such as promoters, operators, terminators and the like, which may be located upstream or downstream of the coding sequence.

The terms “treat,” “treating,” and “treatment” is meant to include alleviating or abrogating a disorder, disease, or condition; or one or more of the symptoms associated with the disorder, disease, or condition; or alleviating or eradicating the cause(s) of the disorder, disease, or condition itself. Desirable effects of treatment can include, but are not limited to, preventing occurrence or recurrence of disease, alleviation of symptoms, diminishing any direct or indirect pathological consequences of the disease, preventing metastasis, decreasing the rate of disease progression, amelioration or palliation of the disease state and remission or improved prognosis.

The term “therapeutically effective amount” refers to the amount of a compound that, when administered, is sufficient to prevent development of, or alleviate to some extent, one or more of the symptoms of the disorder, disease, or condition being treated. The term “therapeutically effective amount” also refers to the amount of a compound that is sufficient to elicit the biological or medical response of a cell, tissue, system, animal, or human that is being sought by a researcher, veterinarian, medical doctor, or clinician.

The term “MYC-driven cancer,” refers to a cancer characterized by aberrant (typically augmented expression) expression of the c-Myc gene or the MYC protein.

### Methods of Diagnosis and Treatment

PVT1, a ‘long non-coding RNA’ adjacent to prominent oncogene c-Myc, has been shown to co-operate with c-Myc by stabilizing its protein product (MYC) in 8q24 gain cancers (Tseng et al. Nature 512, 82-86, 2014, the correlation is illustrated in FIG. 1 of U.S. Publication No. 2021/0071266 A1, which is incorporated by reference herein in its entirety). The present disclosure identifies that targeting PVT1 in 8q24 gain cancers provides a therapeutic window to target MYC, an otherwise notoriously undruggable candidate in cancers. Provided herein in one embodiment are methods of identifying a subject as having a MYC-driven cancer by identifying a PVT1 splice variant in a biological sample isolated from the subject. The PVT1 splice variant comprises PVT1_212, in certain embodiments. The method further comprises identifying a cancer subtype in a subject by detecting a presence or expression level of the PVT1_212 splice variant in a biological sample, such as a liquid sample, a tumor sample, or a non-tumor sample. In some embodiments, c-Myc gene expression level is measured in the biological sample along with detection/measurement of PVT1_212 presence or expression levels. In another embodiment, the copy number of PVT1_212, either alone or in combination with the copy number of c-Myc gene, is identified to diagnose a 8q24 gain MYC-driven cancer. Various methods can be used to identify the PVT1_212 splice variant, such as antibody-based detection, quantitative PCR. Upon detection of the PVT1_212 in the biological sample and assaying its expression level, it is possible to identify the cancer as a MYC-driven cancer. In some embodiments, the cancer is medulloblastoma, and the method disclosed herein enables stratification of the medulloblastoma as Subgroup 3, Subgroup 4, Wnt, or Shh type medulloblastoma based on the expression level of the PVT1_212 splice variant, particularly, the elevated expression level compared to reference values. The reference values are, for instance, in biological samples isolated from a subject who does not have cancer, or a non-tumor sample from the same subject whose tumor sample has elevated PVT1_212. In some embodiments, the expression level of PVT1_212 splice variant is elevated greater than about 2-fold, or about 3-fold, or about 4-fold, or about 5-fold, or about 6-fold, or about 7-fold, or about 8-fold, or about 9-fold, or about 10-fold, or about 11-fold, or about 12-fold, or about 13-fold, or about 14-fold, or about 15-fold, or about 16-fold, or about 17-fold, or about 18-fold, or about 19-fold, or about 20 fold or more compared to a reference value.

It is also identified herein that PVT1_212 undergoes backsplicing and form a circular RNA (CircPVT1_212). Since circular RNAs are more stable than linear RNAs due to their resistance to exonucleases, and can be identified in blood/plasma derived patient samples, this disclosure identifies that, in some cases, the CircPVT1_212 is used as a liquid biopsy marker for MYC-driven, 8q24 gain cancers.

This disclosure further identifies a PVT1_212 splice variant peptide encoded upon circularization (PEPc) and a peptide encoded by the linear form (PEP_{L}). In a further embodiment, several antibodies against the C terminal of PEPc and PEP_{L} which can identify endogenous expression of the PEPs are provided. In some embodiment, these antibodies are used against the PVT variations for histopathology, research, and diagnostic purpose for 8q24 gained, MYC-driven cancers. Further provided are inhibitors against the PEPs (PEPc and PEP_{L}).

Provided herein is a method of detecting a novel PVT-1 splice variant in a biological sample. The method of detection involves: obtaining a biological sample from a subject, isolating a nucleic acid from the biological sample that comprises genomic DNA, and analysis for the presence or absence of the PVT-1 splice variant. In some embodiments, the method comprises detection of a protein product or a peptide encoded by the splice variant that distinguishes the splice variant from the wild type form. In some embodiments such detection involves using an antibody for western hybridization or in situ hybridization detection methods. In some embodiments, the novel PVT-1 splice variant is a circular PVT-1 transcript product. In some embodiments, the novel PVT-1 splice variant is a circular PVT-1 translated product. Identification of the novel PVT-1 splice variant indicates presence of a cMYC driven cancer in the subject.

In some embodiments, identification of any one of the PVT1 splice variants indicated in the above section of the disclosure described herein in a biological sample from a subject is indicative of a MYC-driven cancer in the subject. In some embodiments, identification of Circ PVT1 in a sample from the subject is indicative of a MYC-driven cancer in the subject. In some embodiments one or more PVT1 splice variants can be identified. In some embodiments one or more PVT1 splice variants can be the 104 amino acid PEPc in a biological sample of a subject is indicative of a MYC driven cancer in the subject.

In some embodiments identification of any one or more of the PVT1 splice variant identified in the disclosure is complemented with an additional mode of analysis of a biological sample from the subject for determination of c-MYC driven cancer in a subject.

In some embodiments identification of any one or more of the PVT1 splice variant identified in the disclosure is complemented with one or more physiological evaluations of the subject for determination of c-MYC driven cancer in a subject.

In some embodiments identification of any one or more of the PVT1 splice variant identified in the disclosure is complemented with one or more biochemical evaluations of the subject for determination of c-MYC driven cancer in a subject.

In some embodiments, identification of any one or more of the PVT1 splice variant is performed by analysis of RNA. In some embodiments, qRT-PCR analysis is used for the identification. In some embodiments the identification of any one or more of the PVT1 splice variant is performed by analysis using a PVT1-splice variant specific antibody for determination of c-MYC driven cancer in a subject.

In some embodiments provided herein is a method for treating a subject having a MYC-driven cancer, the method comprising: (a) determining the presence of one or more PVT1 splice variant in a biological sample from the subject, wherein the PVT1-splice variant augments c-MYC expression; (b) administering to the subject a therapeutic composition for the MYC-driven cancer.

Also provided herein is a kit for determining a PVT1 splice variant in a biological sample of a subject.

### EXAMPLES

The examples below further illustrate the described embodiments without limiting the scope of the disclosure.

### Example 1: Identification of the PVT1 Splice Variant Responsible for Stabilizing MYC Protein in Human Cancers

The ENSEMBLE database was searched in order to identify the variants that regulate MYC protein in cancer cells. Accordingly, 25 splice variants of PVT1 have been found (FIG. 2 of U.S. Publication No. 2021/0071266 A1, which is incorporated by reference herein in its entirety). Primers were developed to identify the abundance of each transcript in patient derived medulloblastoma (MB) xenografts (PDX) (FIGS. 3 and 4 of U.S. Publication No. 2021/0071266 A1, which are incorporated by reference herein in their entireties). The analysis suggested that PVT1_212 is the most abundant PVT1 splice variant in all the 4 subgroups of the MB PDXs, while PVT1_203 being the second most prevalent splice variant. PVT1_212 is most prevalent in the MB Subgroup 3 patients, which has the poorest prognosis among the MB patients (FIG. 5 of U.S. Publication No. 2021/0071266 A1, which is incorporated by reference herein in its entirety). Three types of PVT1_212 expression pattern were identified in MB PDXs as well in patient samples: Low PVT1_212 expressing group (0-15×): contained mainly Subgroup 4 MBs, Intermediate PVT1_212 expressing group (15-200×): contained Subgroups 3, Shh and Wnt MB, and the high PVT1_212 expressing group (200-1000×): Exclusively Subgroup 3 (FIGS. 6 and 7 of U.S. Publication No. 2021/0071266 A1, which are incorporated by reference herein in their entireties). This demonstrated that PVT1 expression can be used to stratify MB patients, where the high PVT1_212 expressing group (200-1000×) can designate the 8q24 gain, MYC-driven type of the Group 3 MB patients (generally associated with the poor prognosis).

### Example 2: Functional Identification of the PVT1 Exons Regulating MYC Protein in Human Cancers

For this study, si-RNAs against exon 2, 3 and 9 of the annotated PVT1 gene were designed. Among these, exon 9 exclusively belongs to PVT1_203, whereas exon 2 and 3 is shared between PVT1_212 and PVT1_203. Knock down of PVT1 using si-RNAs against exons 2 and 3, but not Exon 9, reduced MYC protein by as much as 75% (MSTO) to 40% (NCI-H1792) (FIGS. 8, 9, 10, and 11 of U.S. Publication No. 2021/0071266 A1, which are incorporated by reference herein in their entireties). This demonstrated that PVT1_212, and not PVT1_203 was responsible for MYC augmentation in 8q24 gain cancers, and targeting the product of PVT1_212 can significantly reduce the MYC protein levels in these cancers.

### Example 3: PVT1_212 Codes for Novel Peptides that Augment MYC in 8q24 Gain Cancers

This study demonstrated exon 2 of the PVT1 gene can undergo back-splicing and form a circular RNA (CircPVT1_212) (FIGS. 12A-C and 13 of U.S. Publication No. 2021/0071266 A1, which are incorporated by reference herein in their entireties). As shown in FIG. 12A and FIG. 12B(i) of U.S. Publication No. 2021/0071266 A1 PVT1_212 consists of three exons and span over Ch8:127794533-Ch8:127940454. FIG. 12B(ii). cDNA was derived from total RNA obtained from cancer cells using random hexamers (it is important to use random hexamers, since circular RNAs do not have poladenylated 3′ sequence). Divergent primers were designed for each exons of PVT1_212 to identify the CircPVT1 arising from exon 2 of PVT1_212. Sequencing of the CircPVT1 junction confirmed that the junction sequence emanated from the 3′ and 5′ ends of PVT1_212 (FIG. 12B(iii) and FIG. 12C of U.S. Publication No. 2021/0071266 A1). FIG. 12B of U.S. Publication No. 2021/0071266 A1 is a graphical representation indicating that PVT1_212 consists of three exons and span over Ch8:127794533-Ch8:127940454 (FIG. 12B[i] of U.S. Publication No. 2021/0071266 A1). Divergent primers are designed for each exon (FIG. 12B[ii] of U.S. Publication No. 2021/0071266 A1). cDNA was derived from total RNA using random hexamers and sequenced (FIG. 12B[iii] of U.S. Publication No. 2021/0071266 A1). FIG. 12C of U.S. Publication No. 2021/0071266 A1 indicates junction sequence of the CircPVT1 emanating from the 3′ and 5′ ends of PVT1_212 (SEQ ID NO.: 22) (highlighted). Cloning the splice junction demonstrated that this circle emanates from PVT1_212 and not PVT1_203, since the latter starts from ˜150 bp downstream of the beginning of the Exon 2 (FIG. 13 of U.S. Publication No. 2021/0071266 A1, which is incorporated by reference herein in its entirety). FIG. 13 of U.S. Publication No. 2021/0071266 A1 shows the circular PVT1 RNA (SEQ ID NO: 1) in various cell lines. It was found that upon circularization, CircPVT1_212 can form a protein coding ORF of 104 amino acids. The potential peptide was designed as PVT1 Encoded Peptide upon Circularization (PEPc). It was also found that PVT1_212 can encode another peptide (from Exon 1 and 2) of 149 amino acids, which share the same ORF with PEPc for the 94 amino acids at their C terminal end. This variant was designed as PVT1 Encoded Peptide linear form (PEP_{L}) (FIG. 14 of U.S. Publication No. 2021/0071266 A1, which is incorporated by reference herein in its entirety). FIG. 14 of U.S. Publication No. 2021/0071266 A1 is a graphical representation that shows the structure of PEP. PEPc (SEQ ID NO: 33) and PEP_{L} (SEQ ID NO: 34) are shown in FIG. 14 of U.S. Publication No. 2021/0071266 A1. Exogenous addition of PEPc and/or PEP_{L} was seen to augment MYC in U2OS and LNCaP cells, and increase their transformation and metastatic potential (FIGS. 15 and 16 of U.S. Publication No. 2021/0071266 A1, which are incorporated by reference herein in their entireties).

The expression pattern of CircPVT1_212 was investigated in MB PDXs as well as patient samples, and it was observed that the expression of CircPVT1_212 correlates identically with PVT1_212 expression (FIGS. 17, 18, and 19 of U.S. Publication No. 2021/0071266 A1, which are incorporated by reference herein in their entireties). Since circular RNAs are typically more stable than linear RNAs due to their resistance to exonucleases, and can be identified in blood/plasma derived patient samples, this demonstrated that CircPVT1_212 can be used as a liquid biopsy marker for MYC-driven, 8q24 gain cancers. Finally, several antibodies against the C terminal of PEPc and PEP_{L} were derived which can identify endogenous expression of the PEPs (FIGS. 20 and 21 of U.S. Publication No. 2021/0071266 A1, which are incorporated by reference herein in their entireties). Specificity of these antibodies was confirmed by knocking down PVT1_212 (FIG. 21 of U.S. Publication No. 2021/0071266 A1). This demonstrated that the antibodies against PEPs can be used for histopathology, research and diagnostic purpose for 8q24 gained, MYC-driven cancer. FIG. 22 of U.S. Publication No. 2021/0071266 A1 shows gain of 8q24 is a common amplified region in human cancer. In an exemplary assay, siRNAs that can bind to Exons 2, 3, 9, 20 and 30 of PVT1 were separately expressed in cancer cell lines MSTO and NCIH1792. It was observed that only siRNA directed to the Exon 2 and Exon 2 showed remarkable reduction in MYC protein levels (FIG. 23 of U.S. Publication No. 2021/0071266 A1, which is incorporated by reference herein in its entirety). FIG. 24 of U.S. Publication No. 2021/0071266 A1, which is incorporated by reference herein in its entirety, shows effect of siRNAs against PVT1 exons on mRNA expression of Myc in MSTO and NCIH1792 cells. FIG. 25 of U.S. Publication No. 2021/0071266 A1, which is incorporated by reference herein in its entirety, shows splice junction between exons 1 and 2 of PVT1. A portion of PVT1_212 is shown below (SEQ ID NO: 36) in FIG. 25 of U.S. Publication No. 2021/0071266 A1. The amplicon (88 bp; SEQ ID NO: 45) is spanning PVT1 exon 1-2 junction, which contains Ex12-F1 (SEQ ID NO: 35) in exon 1 and Ex12-R1 (SEQ ID NO; 37) in exon 2 as shown in FIG. 25 of U.S. Publication No. 2021/0071266 A1. Expression of Exons 1 and 2 were determined by RT-qPCR on Medulloblastoma (MedullB) samples, as shown in FIG. 26 of U.S. Publication No. 2021/0071266 A1, which is incorporated by reference herein in its entirety. The splice variants are PCR amplified with suitable primers and cloned in Topo vectors for sequence analysis as shown in FIGS. 27-29 of U.S. Publication No. 2021/0071266 A1, which are incorporated by reference herein in their entireties. FIG. 28 of U.S. Publication No. 2021/0071266 A1 shows sequence analysis of highest expression variants (For Clone M4, SEQ ID NOS 38-52, from line 1 to line 15, respectively; for Clone M5, SEQ ID NOs: 53 to 67, from line 1 to line 15, respectively). FIG. 29 of U.S. Publication No. 2021/0071266 A1 shows sequence analysis of highest expression variant (For Clone 23, SEQ ID NOS 68-80, from line 1 to line 13, respectively; for Clone 1377, SEQ ID NOs: 81 to 95, from line 1 to line 15, respectively).

### Example 4: Circ PVT1 in MYC Cancers

Expression of the CircPVT1 was further investigated in patient tissue samples. CircPVT1 is enriched in MYC driven Group 3 medulloblastoma tumors. FIG. 30A of U.S. Publication No. 2021/0071266 A1, which is incorporated by reference herein in its entirety, subgroup of Group 3 medulloblastoma harbor MYC amplification and have the worst clinical outcome compared to the other medulloblastoma groups (Cavalli et al, Cancer Cell, 31:737-754). FIG. 30B of U.S. Publication No. 2021/0071266 A1, which is incorporated by reference herein in its entirety, shows results from quantitative RT-PCR analysis of patient derived xenografts (PDX samples) from medulloblastoma patients (obtained from Dr. Weschler-Reya's lab at SBP). The results revealed that the CircPVT1 is especially enriched in the MYC driven Group 3 medulloblastoma tumors.

High abundance of CircPVT1 is correlated to high MYC protein in multiple cancer cell lines. In order to examine whether abundance of CircPVT1 and MYC are correlated in multiple cancer cell lines, 4 cell lines were selected with high copy number of MYC and PVT1 (Hi-MYC cell lines: PSN-1, NCIH-2170, NCIH-1792, MSTO-211H) and 4 cell lines with MYC+PVT1 copy number neutral cell lines (Lo-MYC cell lines: U2OS, BxPC-3, DU145 and PC-3) from ATCC. These 8 cell lines represent a broad array of different types of cancers, as outlined in FIG. 31A of U.S. Publication No. 2021/0071266 A1, which is incorporated by reference herein in its entirety. It was confirmed that the Hi-MYC cell lines are enriched in MYC protein, compared to the MYC protein in Lo-MYC cell lines (FIG. 31B of U.S. Publication No. 2021/0071266 A1, which is incorporated by reference herein in its entirety). FIG. 31C of U.S. Publication No. 2021/0071266 A1, which is incorporated by reference herein in its entirety, shows results from q-RT-PCR which revealed that CircPVT1 is expressed in much higher levels in Hi-MYC cell lines compared to those in Lo-MYC cell lines.

Inhibition of CircPVT1 leads to growth arrest of Hi- and Lo-MYC cell lines. si-RNA was designed (si_C2) against the junction of the CircPVT1 which inhibits the expression of CircPVT1, but not that of PVT1_212 (FIG. 32A of U.S. Publication No. 2021/0071266 A1, which is incorporated by reference herein in its entirety). Cell proliferation was assessed by counting cells following the transfection of the Hi-MYC and Lo-MYC cell lines with si_C2 (in red, also indicated by an arrow) and control si (si_Ctr, in blue) over the indicated period in the X-axis in in FIG. 32B of U.S. Publication No. 2021/0071266 A1, which is incorporated by reference herein in its entirety. In each case, the siRNA directed to CircPVT1(si_C2) inhibited proliferation of the MYC cell lines. The data demonstrates that the expression of the CircPVT1 is necessary for the proliferation of Hi-MYC as well as Lo-MYC cell lines, thus establishing the centrality of the CircPVT1 for the proliferation of cancer cells.

Next, it was investigated whether CircPVT1 can regulate MYC levels. Indeed, siRNA mediated knockdown of CircPVT1 in Hi- and Lo-MYC cell lines resulted in reduction in the MYC level in Hi-MYC as well as Lo-MYC cells, suggesting that CircPVT1 is required for MYC protein levels in cancer cells (FIG. 33 of U.S. Publication No. 2021/0071266 A1, which is incorporated by reference herein in its entirety).

It was found that circularization of RNA from Exon 2 of PVT1_212 reorganizes its reading frame, resulting into a novel open reading frame coding for 104 amino acids containing peptide, henceforth known as PEPc (PVT1 Encoded Peptide upon Circularization) or Conjoined (CJN). FIG. 34A of U.S. Publication No. 2021/0071266 A1, which is incorporated by reference herein in its entirety shows a schematic diagram of the circularized of RNA with the open reading frame indicated, with the start codon (ATG) and the stop codon (TGA), encoding PEPc. A monoclonal antibody was developed against PEPc/CJN which can detect endogenously expressed PEPc/CJN in the four Hi-MYC cell lines (FIG. 34B of U.S. Publication No. 2021/0071266 A1, which is incorporated by reference herein in its entirety). The specificity of the antibody was confirmed by carrying out a Western Blot analysis where inhibition of the CircPVT1 (by si_C2) but not of PVT1_212 (by si_3) resulted into reduction of the protein detected by the PEPc antibody (FIG. 34C of U.S. Publication No. 2021/0071266 A1, which is incorporated by reference herein in its entirety).

Expression of PEPc is sufficient to augment MYC in cancer cells. Western blot analysis of MYC protein in U2OS cells stably transfected with Luciferase (as control) or PEPc, showed increase in MYC levels on ectopic expression of PEPc (FIG. 35A of U.S. Publication No. 2021/0071266 A1, which is incorporated by reference herein in its entirety). PEPc expression increased colony formation and migration ability of U2OS (FIG. 35B of U.S. Publication No. 2021/0071266 A1, which is incorporated by reference herein in its entirety). Soft agar assay showed an increase in colony numbers compared to the control when equal number of U2OS+Luciferase and U2OS+PEPc cells are plated on soft agar. Transwell migration assay showed increase in migration potential in U2OS+PEPc cells.

Additional sequences that may be used in the methods disclosed herein include those listed in Table 1, below.

| SEQ | | |
|---|---|---|
| ID | | |
| NO. | Sequence | Description |
| 1 | GCCTGATCTTTTGGCCAGAAGGAGATTAAAAAGATGCCC | Circular RNA of |
| | CTCAAGATGGCTGTGCCTGTCAGCTGCATGGAGCTTCGT | exon 2 of PVT1 |
| | TCAAGTATTTTCTGAGCCTGATGGATTTACAGTGATCTT | splice variant |
| | CAGTGGTCTGGGGAATAACGCTGGTGGAACCATGCACTG | PVT1_212 |
| | GAATGACACACGCCCGGCACATTTCAGGATACTAAAAGT | (“CircPVT1”) |
| | GGTTTTAAGGGAGGCTGTGGCTGAATGCCTCATGGATTC | |
| | TTACAGCTTGGATGTCCATGGGGGACGAAGGACTGCAGC | |
| | TGGCTGAGAGGGTTGAGATCTCTGTTTACTTAGATCTCT | |
| | GCCAACTTCCTTTGGGTCTCCCTATGGAATGTAAGACCC | |
| | CGACTCTTCCTGGTGAAGCATCTGATGCACGTTCCATCC | |
| | GGCGCTCAGCTGGGCTTGAG | |
| 2 | CCATCCGGCGCTCAG | Si_CircPVT1 or |
| | | si_C2: Target |
| | | sequence |
| 3 | UGGGCUUGAGGCCUGAUCUUU | Sense sequence |
| | | for Si_CircPVT1 |
| | | or si_C2: Target |
| | | sequence |
| 4 | AGAUCAGGCCUCAAGCCCAUU | Antisense |
| | | sequence for |
| | | Si_CircPVT1 or |
| | | si_C2: Target |
| | | sequence |
| 5 | GCCATCATGATGGTACTTT | siRNA PVT1 |
| | | Exon 3 target |
| | | sequence |
| 6 | CAUCAUGAUGGUACU U UAATT | siRNA PVT1 |
| | | Exon 3 sense |
| | | strand |
| 7 | UUAAAGUACCAUCAUGAUGGC | siRNA PVT1 |
| | | Exon 3 antisense |
| | | strand |
| 8 | CCGGCACATTTCAGGATACTA | siRNA PVT1 |
| | | Exon 2 target |
| | | sequence |
| 9 | GGCACAUUUCAGGAUACUATT | siRNA PVT1 |
| | | Exon 2 target |
| | | sequence |
| 10 | UAGUAUCCUGAMUGUGCCGG | siRNA PVT1 |
| | | Exon 2 target |
| | | sequence |
| 11 | atgcacgttccatcc | CircPVT1's |
| | ggcgctcagctgggcttgaggcctgatcttttggccagaaggaga | endogenously |
| | ttaaaaagatgcccctcaagatggctgtgcctgtcagctgcatgg | expressed 104 aa |
| | agcttcgttcaagtattttctgagcctgatggatttacagtgatc | peptide (“PEPc” |
| | ttcagtggtctggggaataacgctggtggaaccatgcactggaat | or “Conjoined”) |
| | gacacacgcccggcacatttcaggatactaaaagtggttttaagg | |
| | gaggctgtggctgaatgcctcatggattcttacagcttggatgtc | |
| | catgggggacgaaggactgcagctggctga | |
| 12 | M H V P S G A Q L G L R P D L | CircPVT1's |
| | L A R R R L K R C P S R W L C | endogenously |
| | L S A A W S F V Q V F S E P D | expressed 104 aa |
| | G F T V I F S G L G N N A G G | peptide (“PEPc” |
| | T M H W N D T R P A H F R I L | or “Conjoined”) |
| | K V V L R E A V A E C L M D S | |
| | Y S L D V H G G R R T A A G * | |
| 13 | ATG CAC GTT CCA TCC GGC GCT CAG CTG GGC TTG | Original PEPc |
| | AGG CCT GAT CTT TTG GCC AGA AGG AGA TTA AAA | DNA |
| | AGA TGC CCC TCA AGA TGG CTG TGC CTG TCA GCT | |
| | GCA TGG AGC TTC GTT CAA GTA TTT TCT GAG CCT | |
| | GAT GGA TTT ACA GTG ATC TTC AGT GGT CTG GGG | |
| | AAT AAC GCT GGT GGA ACC ATG CAC TGG AAT GAC | |
| | ACA CGC CCG GCA CAT TTC AGG ATA CTA AAA GTG | |
| | GTT TTA AGG GAG GCT GTG GCT GAA TGC CTC ATG | |
| | GAT TCT TAC AGC TTG GAT GTC CAT GGG GGA CGA | |
| | AGG ACT GCA GCT GGC TGA | |
| 14 | MHVPSGAQLGLRPDLLARRRLKRCPSRWLCLSAAWSFVQV | Original PEPc aa |
| | FSEPDGFTVIFSGLGNNAGGTMHWNDTRPAHFRILKVVLR | (104 aa) |
| | EAVAECLMDSYSLDVHGGRRTAAG | |
| 15 | ATG CAT GTA CCT TCC GGC GCC CAA CTC GGC CTC | Codon optimized |
| | AGA CCG GAC CTG TTG GCC CGA CGA CGA CTG AAG | PEPc DNA |
| | CGA TGC CCT AGC AGG TGG CTC TGT CTG TCA GCT | |
| | GCG TGG TCT TTT GTC CAA GTT TTC TCC GAG CCA | |
| | GAT GGT TTC ACA GTT ATT TTC TCC GGG TTG GGT | |
| | AAC AAT GCG GGC GGC ACT ATG CAT TGG AAT GAT | |
| | ACT AGA CCA GCA CAC TTT AGG ATC TTG AAA GTG | |
| | GTC CTC AGG GAA GCG GTG GCG GAA TGT CTG ATG | |
| | GAT AGT TAT TCA CTG GAC GTA CAT GGG GGT CGC | |
| | CGA ACA GCC GCA GGC TGA | |
| 16 | TGA CAC GTT CCA TCC GGC GCT CAG CTG GGC TTG | PEPc (ATG > |
| | AGG CCT GAT CTT TTG GCC AGA AGG AGA TTA AAA | TGA) DNA |
| | AGA TGC CCC TCA AGA TGG CTG TGC CTG TCA GCT | |
| | GCA TGG AGC TTC GTT CAA GTA TTT TCT GAG CCT | |
| | GAT GGA TTT ACA GTG ATC TTC AGT GGT CTG GGG | |
| | AAT AAC GCT GGT GGA ACC TGA CAC TGG AAT GAC | |
| | ACA CGC CCG GCA CAT TTC AGG ATA CTA AAA GTG | |
| | GTT TTA AGG GAG GCT GTG GCT GAA TGC CTC TGA | |
| | GAT TCT TAC AGC TTG GAT GTC CAT GGG GGA CGA | |
| | AGG ACT GCA GCT GGC TGA | |

While preferred embodiments of the present disclosure have been shown and described herein, it will be obvious to those skilled in the art that such embodiments are provided by way of example only. It is not intended that the disclosed methods be limited by the specific examples provided within the specification. While the disclosed methods have been described with reference to the aforementioned specification, the descriptions and illustrations of the embodiments herein are not meant to be construed in a limiting sense. Numerous variations, changes, and substitutions will now occur to those skilled in the art without departing from the disclosure. Furthermore, it shall be understood that all aspects of the disclosure are not limited to the specific depictions, configurations or relative proportions set forth herein which depend upon a variety of conditions and variables. It should be understood that various alternatives to the embodiments of the disclosed methods described herein may be employed in practicing the methods. It is therefore contemplated that the disclosure shall also cover any such alternatives, modifications, variations or equivalents. It is intended that the following claims define the scope of the disclosure and that methods and structures within the scope of these claims and their equivalents be covered thereby.

What is claimed is:

1. A method of treating a cancer in a subject, the method comprising administering a therapy targeting a peptide encoded by a PVT1 splice variant PVT1_212, wherein the subject has previously been identified as having a cancer characterized by co-gain of PVT1 and c-Myc, wherein said peptide comprises a PEPc peptide comprising the sequence set forth in SEQ ID NO. 14, or a PEP_{L} peptide comprising the sequence set forth in SEQ ID NO. 34.

2. The method of claim 1, wherein the co-gain is identified by assaying copy numbers of PVT1 and c-Myc in a biological sample isolated from said subject and comparing the copy numbers to reference values.

3. The method of claim 2, wherein the reference values are the copy numbers of PVT1 and c-Myc in a biological sample isolated from a subject who does not have a cancer.

4. The method of claim 2, wherein the reference values are the copy numbers of PVT1 and c-Myc in a non-tumor sample isolated from said subject.

5. The method of claim 1, wherein the co-gain is identified by assaying copy numbers of PVT1 and c-Myc in a tumor sample isolated from said subject and comparing with copy numbers of said genes available from the TCGA or ENSEMBL database.

6. The method of claim 1, wherein the cancer comprises a 8q24.21 gain cancer.

7. The method of claim 1, wherein the cancer comprises medulloblastoma, breast cancer, ovarian cancer, lung cancer, prostate cancer, or a colorectal cancer.


SEQUENCE LISTING (95 sequences)

SEQ ID NO: 1 - DNA, 410 bp, Homo sapiens
  source 1..410: mol_type = unassigned DNA
gcctgatcttttggccagaaggagattaaaaagatgcccctcaagatggctgtgcctgtcagctgcatggagcttcgttcaagtattttctgagcctgatggatttacagtgatcttcagtggtctggggaataacgctggtggaaccatgcactggaatgacacacgcccggcacatttcaggatactaaaagtggttttaagggaggctgtggctgaatgcctcatggattcttacagcttggatgtccatgggggacgaaggactgcagctggctgagagggttgagatctctgtttacttagatctctgccaacttcctttgggtctccctatggaatgtaagaccccgactcttcctggtgaagcatctgatgcacgttccatccggcgctcagctgggcttgag

SEQ ID NO: 2 - DNA, 15 bp, synthetic construct
  misc_feature 1..15: note = Description of Artificial Sequence: Synthetic oligonucleotide
  source 1..15: mol_type = other DNA
ccatccggcgctcag

SEQ ID NO: 3 - RNA, 21 nt, synthetic construct
  misc_feature 1..21: note = Description of Artificial Sequence: Synthetic oligonucleotide
  source 1..21: mol_type = other RNA
tgggcttgaggcctgatcttt

SEQ ID NO: 4 - RNA, 21 nt, synthetic construct
  misc_feature 1..21: note = Description of Artificial Sequence: Synthetic oligonucleotide
  source 1..21: mol_type = other RNA
agatcaggcctcaagcccatt

SEQ ID NO: 5 - DNA, 19 bp, synthetic construct
  misc_feature 1..19: note = Description of Artificial Sequence: Synthetic oligonucleotide
  source 1..19: mol_type = other DNA
gccatcatgatggtacttt

SEQ ID NO: 6 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
catcatgatggtactttaatt

SEQ ID NO: 7 - RNA, 21 nt, synthetic construct
  misc_feature 1..21: note = Description of Artificial Sequence: Synthetic oligonucleotide
  source 1..21: mol_type = other RNA
ttaaagtaccatcatgatggc

SEQ ID NO: 8 - DNA, 21 bp, synthetic construct
  misc_feature 1..21: note = Description of Artificial Sequence: Synthetic oligonucleotide
  source 1..21: mol_type = other DNA
ccggcacatttcaggatacta

SEQ ID NO: 9 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ggcacatttcaggatactatt

SEQ ID NO: 10 - RNA, 20 nt, synthetic construct
  misc_feature 1..20: note = Description of Artificial Sequence: Synthetic oligonucleotide
  source 1..20: mol_type = other RNA
tagtatcctgamtgtgccgg

SEQ ID NO: 11 - DNA, 315 bp, Homo sapiens
  source 1..315: mol_type = unassigned DNA
atgcacgttccatccggcgctcagctgggcttgaggcctgatcttttggccagaaggagattaaaaagatgcccctcaagatggctgtgcctgtcagctgcatggagcttcgttcaagtattttctgagcctgatggatttacagtgatcttcagtggtctggggaataacgctggtggaaccatgcactggaatgacacacgcccggcacatttcaggatactaaaagtggttttaagggaggctgtggctgaatgcctcatggattcttacagcttggatgtccatgggggacgaaggactgcagctggctga

SEQ ID NO: 12 - protein, 104 aa, Homo sapiens
MHVPSGAQLGLRPDLLARRRLKRCPSRWLCLSAAWSFVQVFSEPDGFTVIFSGLGNNAGGTMHWNDTRPAHFRILKVVLREAVAECLMDSYSLDVHGGRRTAAG

SEQ ID NO: 13 - DNA, 315 bp, Homo sapiens; identical to SEQ ID NO: 11
  source 1..315: mol_type = unassigned DNA

SEQ ID NO: 14 - protein, 104 aa, Homo sapiens; identical to SEQ ID NO: 12

SEQ ID NO: 15 - DNA, 315 bp, synthetic construct
  misc_feature 1..315: note = Description of Artificial Sequence: Synthetic polynucleotide
  source 1..315: mol_type = other DNA
atgcatgtaccttccggcgcccaactcggcctcagaccggacctgttggcccgacgacgactgaagcgatgccctagcaggtggctctgtctgtcagctgcgtggtcttttgtccaagttttctccgagccagatggtttcacagttattttctccgggttgggtaacaatgcgggcggcactatgcattggaatgatactagaccagcacactttaggatcttgaaagtggtcctcagggaagcggtggcggaatgtctgatggatagttattcactggacgtacatgggggtcgccgaacagccgcaggctga

SEQ ID NO: 16 - DNA, 315 bp, synthetic construct
  misc_feature 1..315: note = Description of Artificial Sequence: Synthetic polynucleotide
  source 1..315: mol_type = other DNA
tgacacgttccatccggcgctcagctgggcttgaggcctgatcttttggccagaaggagattaaaaagatgcccctcaagatggctgtgcctgtcagctgcatggagcttcgttcaagtattttctgagcctgatggatttacagtgatcttcagtggtctggggaataacgctggtggaacctgacactggaatgacacacgcccggcacatttcaggatactaaaagtggttttaagggaggctgtggctgaatgcctctgagattcttacagcttggatgtccatgggggacgaaggactgcagctggctga

SEQ ID NO: 17 - DNA, 48 bp, synthetic construct
  misc_feature 1..48: note = Description of Artificial Sequence: Synthetic oligonucleotide
  source 1..48: mol_type = other DNA
cggcgctcagctgggcttgaggcctgatcttttggccagaaggagatt

SEQ ID NO: 18 - DNA, 387 bp, Homo sapiens
  source 1..387: mol_type = unassigned DNA
gcctgatcttttggccagaaggagattaaaaagatgcccctcaagatggctgtgcctgtcagctgcatggagcttcgttcaagtattttctgagcctgatggatttacagtgatcttcagtggtctggggaataacgctggtggaaccatgcactggaatgacacacgcccggcacatttcaggatactaaaagtggttttaagggaggctgtggctgaatgcctcatggattcttacagcttggatgtccatgggggacgaaggactgcagctggctgagagggttgagatctctgtttacttagatctctgccaacttcctttgggtctccctatggaatgtaagaccccgactcttcctggtgaagcatctgatgcacgttcca

SEQ ID NO: 19 - DNA, 344 bp, Homo sapiens
  source 1..344: mol_type = unassigned DNA
gcctgatcttttggccagaaggagattaaaaagatgcccctcaagatggctgtgcctgtcagctgcatggagcttcgttcaagtattttctgagcctgatggatttacagtgatcttcagtggtctggggaataacgctggtggaaccatgcactggaatgacacacgcccggcacatttcaggatactaaaagtggttttaagggaggctgtggctgaatgcctcatggattcttacagcttggatgtccatgggggacgaaggactgcagctggctgagagggttgagatctctgtttacttagatctctgccaacttcctttgggtctccctatggaatgt

SEQ ID NO: 20 - DNA, 372 bp, Homo sapiens
  source 1..372: mol_type = unassigned DNA
gcctgatcttttggccagaaggagattaaaaagatgcccctcaagatggctgtgcctgtcagctgcatggagcttcgttcaagtattttctgagcctgatggatttacagtgatcttcagtggtctggggaataacgctggtggaaccatgcactggaatgacacacgcccggcacatttcaggatactaaaagtggttttaagggaggctgtggctgaatgcctcatggattcttacagcttggatgtccatgggggacgaaggactgcagctggctgagagggttgagatctctgtttacttagatctctgccaacttcctttgggtctccctatggaatgtaagaccccgactcttcctggtgaagcat

SEQ ID NO: 21 - DNA, 153 bp, Homo sapiens
  source 1..153: mol_type = unassigned DNA
gcctgatcttttggccagaaggagattaaaaagatgcccctcaagatggctgtgcctgtcagctgcatggagcttcgttcaagtattttctgagcctgatggatttacagtgatcttcagtggtctggggaataacgctggtggaaccatgca

SEQ ID NO: 22 - DNA, 410 bp, Homo sapiens; identical to SEQ ID NO: 1
  source 1..410: mol_type = unassigned DNA

SEQ ID NO: 23 - DNA, 207 bp, Homo sapiens
  source 1..207: mol_type = unassigned DNA
gcctgatcttttggccagaaggagattaaaaagatgcccctcaagatggctgtgcctgtcagctgcatggagcttcgttcaagtattttctgagcctgatggatttacagtgatcttcagtggtctggggaataacgctggtggaaccatgcactggaatgacacacgcccggcacatttcaggatactaaaagtggttttaaggga

SEQ ID NO: 24 - DNA, 395 bp, Homo sapiens
  source 1..395: mol_type = unassigned DNA
gcctgatcttttggccagaaggagattaaaaagatgcccctcaagatggctgtgcctgtcagctgcatggagcttcgttcaagtattttctgagcctgatggatttacagtgatcttcagtggtctggggaataacgctggtggaaccatgcactggaatgacacacgcccggcacatttcaggatactaaaagtggttttaagggaggctgtggctgaatgcctcatggattcttacagcttggatgtccatgggggacgaaggactgcagctggctgagagggttgagatctctgtttacttagatctctgccaacttcctttgggtctccctatggaatgtaagaccccgactcttcctggtgaagcatctgatgcacgttccatccggcgc

SEQ ID NO: 25 - DNA, 364 bp, Homo sapiens
  source 1..364: mol_type = unassigned DNA
gcctgatcttttggccagaaggagattaaaaagatgcccctcaagatggctgtgcctgtcagctgcatggagcttcgttcaagtattttctgagcctgatggatttacagtgatcttcagtggtctggggaataacgctggtggaaccatgcactggaatgacacacgcccggcacatttcaggatactaaaagtggttttaagggaggctgtggctgaatgcctcatggattcttacagcttggatgtccatgggggacgaaggactgcagctggctgagagggttgagatctctgtttacttagatctctgccaacttcctttgggtctccctatggaatgtaagaccccgactcttcctgg

SEQ ID NO: 26 - DNA, 371 bp, Homo sapiens
  source 1..371: mol_type = unassigned DNA
ctcaagatggctgtgcctgtcagctgcatggagcttcgttcaagtattttctgagcctgatggatttacagtgatcttcagtggtctggggaataacgctggtggaaccatgcactggaatgacacacgcccggcacatttcaggatactaaaagtggttttaagggaggctgtggctgaatgcctcatggattcttacagcttggatgtccatgggggacgaaggactgcagctggctgagagggttgagatctctgtttacttagatctctgccaacttcctttgggtctccctatggaatgtaagaccccgactcttcctggtgaagcatctgatgcacgttccatccggcgctcagctgggcttgag

SEQ ID NO: 27 - DNA, 129 bp, Homo sapiens
  source 1..129: mol_type = unassigned DNA
agggttgagatctctgtttacttagatctctgccaacttcctttgggtctccctatggaatgtaagaccccgactcttcctggtgaagcatctgatgcacgttccatccggcgctcagctgggcttgag

SEQ ID NO: 28 - DNA, 322 bp, Homo sapiens
  source 1..322: mol_type = unassigned DNA
tctgagcctgatggatttacagtgatcttcagtggtctggggaataacgctggtggaaccatgcactggaatgacacacgcccggcacatttcaggatactaaaagtggttttaagggaggctgtggctgaatgcctcatggattcttacagcttggatgtccatgggggacgaaggactgcagctggctgagagggttgagatctctgtttacttagatctctgccaacttcctttgggtctccctatggaatgtaagaccccgactcttcctggtgaagcatctgatgcacgttccatccggcgctcagctgggcttgag

SEQ ID NO: 29 - DNA, 353 bp, Homo sapiens
  source 1..353: mol_type = unassigned DNA
gtcagctgcatggagcttcgttcaagtattttctgagcctgatggatttacagtgatcttcagtggtctggggaataacgctggtggaaccatgcactggaatgacacacgcccggcacatttcaggatactaaaagtggttttaagggaggctgtggctgaatgcctcatggattcttacagcttggatgtccatgggggacgaaggactgcagctggctgagagggttgagatctctgtttacttagatctctgccaacttcctttgggtctccctatggaatgtaagaccccgactcttcctggtgaagcatctgatgcacgttccatccggcgctcagctgggcttgag

SEQ ID NO: 30 - DNA, 10 bp, Homo sapiens
  source 1..10: mol_type = unassigned DNA
cctgatcttt

SEQ ID NO: 31 - DNA, 10 bp, Homo sapiens
  source 1..10: mol_type = unassigned DNA
gggcttgagg

SEQ ID NO: 32 - DNA, 25 bp, synthetic construct
  misc_feature 1..25: note = Description of Artificial Sequence: Synthetic oligonucleotide
  source 1..25: mol_type = other DNA
gctgggcttgaggcctgatcttttg

SEQ ID NO: 33 - protein, 104 aa, Homo sapiens; identical to SEQ ID NO: 12

SEQ ID NO: 34 - protein, 149 aa, Homo sapiens
MGPRAGRARGGRDEEGRRRAASKDVPRDPRHLPVDFLAERMLAVPVTCGDTARSALQPDLLARRRLKRCPSRWLCLSAAWSFVQVFSEPDGFTVIFSGLGNNAGGTMHWNDTRPAHFRILKVVLREAVAECLMDSYSLDVHGGRRTAAG

SEQ ID NO: 35 - DNA, 20 bp, synthetic construct
  misc_feature 1..20: note = Description of Artificial Sequence: Synthetic primer
  source 1..20: mol_type = other DNA
cctgtgacctgtggagacac

SEQ ID NO: 36 - DNA, 140 bp, Homo sapiens
  source 1..140: mol_type = unassigned DNA
cttgcggaaaggatgttggcggtccctgtgacctgtggagacacggccagatctgccctccagcctgatcttttggccagaaggagattaaaaagatgcccctcaagatggctgtgcctgtcagctgcatggagcttcgt

SEQ ID NO: 37 - DNA, 20 bp, synthetic construct
  misc_feature 1..20: note = Description of Artificial Sequence: Synthetic primer
  source 1..20: mol_type = other DNA
gccatcttgaggggcatctt

SEQ ID NO: 38 - DNA, 165 bp, synthetic construct
  misc_feature 1..165: note = Description of Artificial Sequence: Synthetic polynucleotide
  source 1..165: mol_type = other DNA
aatacgactcactatagggcgaattgaatttagcggccgcgaattcgcccttcctgtgacctgtggagacacggccagatctgccctccagcctgatcttttggccagaaggagattaaaaagatgcccctcaagatggcaagggcgaattcgtttaaacctgca

SEQ ID NO: 39 - DNA, 140 bp, synthetic construct
  misc_feature 1..140: note = Description of Artificial Sequence: Synthetic polynucleotide
  source 1..140: mol_type = other DNA
aatacgactcactatagggcgaattgaatttagcggccgcgaattcgcccttcctgtgacctgtggagacacggccagatctgccctccagcctgatcttttggccagaaggagattaaaaagatgcccctcaagatggc

SEQ ID NO: 40 - DNA, 140 bp, synthetic construct; identical to SEQ ID NO: 39
  misc_feature 1..140: note = Description of Artificial Sequence: Synthetic polynucleotide
  source 1..140: mol_type = other DNA

SEQ ID NO: 41 - DNA, 165 bp, synthetic construct; identical to SEQ ID NO: 38
  misc_feature 1..165: note = Description of Artificial Sequence: Synthetic polynucleotide
  source 1..165: mol_type = other DNA

SEQ ID NO: 42 - DNA, 140 bp, synthetic construct; identical to SEQ ID NO: 39
  misc_feature 1..140: note = Description of Artificial Sequence: Synthetic polynucleotide
  source 1..140: mol_type = other DNA

SEQ ID NO: 43 - DNA, 18 bp, synthetic construct
  misc_feature 1..18: note = Description of Artificial Sequence: Synthetic oligonucleotide
  source 1..18: mol_type = other DNA
aatacgactcactatagg

SEQ ID NO: 44 - DNA, 165 bp, synthetic construct; identical to SEQ ID NO: 38
  misc_feature 1..165: note = Description of Artificial Sequence: Synthetic polynucleotide
  source 1..165: mol_type = other DNA

SEQ ID NO: 45 - DNA, 88 bp, synthetic construct
  misc_feature 1..88: note = Description of Artificial Sequence: Synthetic oligonucleotide
  source 1..88: mol_type = other DNA
cctgtgacctgtggagacacggccagatctgccctccagcctgatcttttggccagaaggagattaaaaagatgcccctcaagatggc

SEQ ID NO: 46 - DNA, 135 bp, synthetic construct
  misc_feature 1..135: note = Description of Artificial Sequence: Synthetic polynucleotide
  source 1..135: mol_type = other DNA
tagcggccgcgaattcgcccttcctgtgacctgtggagacacggccagatctgccctccagcctgatcttttggccagaaggagattaaaaagatgcccctcaagatggcaagggcgaattcgtttaaacctgca

SEQ ID NO: 47 - DNA, 135 bp, synthetic construct; identical to SEQ ID NO: 46
  misc_feature 1..135: note = Description of Artificial Sequence: Synthetic polynucleotide
  source 1..135: mol_type = other DNA

SEQ ID NO: 48 - DNA, 133 bp, synthetic construct
  misc_feature 1..133: note = Description of Artificial Sequence: Synthetic polynucleotide
  source 1..133: mol_type = other DNA
gcggccgcgaattcgcccttcctgtgacctgtggagacacggccagatctgccctccagcctgatcttttggccagaaggagattaaaaagatgcccctcaagatggcaagggcgaattcgtttaaacctgca

SEQ ID NO: 49 - DNA, 83 bp, synthetic construct
  misc_feature 1..83: note = Description of Artificial Sequence: Synthetic oligonucleotide
  source 1..83: mol_type = other DNA
gccctccagcctgatcttttggccagaaggagattaaaaagatgcccctcaagatggcaagggcgaattcgtttaaacctgca

SEQ ID NO: 50 - DNA, 68 bp, synthetic construct
  misc_feature 1..68: note = Description of Artificial Sequence: Synthetic oligonucleotide
  source 1..68: mol_type = other DNA
cttttggccagaaggagattaaaaagatgcccctcaagatggcaagggcgaattcgtttaaacctgca

SEQ ID NO: 51 - DNA, 66 bp, synthetic construct
  misc_feature 1..66: note = Description of Artificial Sequence: Synthetic oligonucleotide
  source 1..66: mol_type = other DNA
tttggccagaaggagattaaaaagatgcccctcaagatggcaagggcgaattcgtttaaacctgca

SEQ ID NO: 52 - DNA, 66 bp, synthetic construct; identical to SEQ ID NO: 51
  misc_feature 1..66: note = Description of Artificial Sequence: Synthetic oligonucleotide
  source 1..66: mol_type = other DNA

SEQ ID NO: 53 - DNA, 137 bp, synthetic construct
  misc_feature 1..137: note = Description of Artificial Sequence: Synthetic polynucleotide
  source 1..137: mol_type = other DNA
tttagcggccgcgaattcgcccttcctgtgacctgtggagacacggccagatctgccctccagcctgatcttttggccagaaggagattaaaaagatgcccctcaagatggcaagggcgaattcgtttaaacctgca

SEQ ID NO: 54 - DNA, 137 bp, synthetic construct; identical to SEQ ID NO: 53
  misc_feature 1..137: note = Description of Artificial Sequence: Synthetic polynucleotide
  source 1..137: mol_type = other DNA

SEQ ID NO: 55 - DNA, 137 bp, synthetic construct
  misc_feature 1..137: note = Description of Artificial Sequence: Synthetic polynucleotide
  source 1..137: mol_type = other DNA
tttagcggccgcgaattcgcccttcctgtgacctgtggagacacggccagatctgccctccagcctgatcttttggccagaaagagattaaaaagatgcccctcaagatggcaagggcgaattcgtttaaacctgca

SEQ ID NO: 56 - DNA, 137 bp, synthetic construct; identical to SEQ ID NO: 53
  misc_feature 1..137: note = Description of Artificial Sequence: Synthetic polynucleotide
  source 1..137: mol_type = other DNA

SEQ ID NO: 57 - DNA, 137 bp, synthetic construct; identical to SEQ ID NO: 53
  misc_feature 1..137: note = Description of Artificial Sequence: Synthetic polynucleotide
  source 1..137: mol_type = other DNA

SEQ ID NO: 58 - DNA, 137 bp, synthetic construct; identical to SEQ ID NO: 53
  misc_feature 1..137: note = Description of Artificial Sequence: Synthetic polynucleotide
  source 1..137: mol_type = other DNA

SEQ ID NO: 59 - DNA, 137 bp, synthetic construct; identical to SEQ ID NO: 53
  misc_feature 1..137: note = Description of Artificial Sequence: Synthetic polynucleotide
  source 1..137: mol_type = other DNA

SEQ ID NO: 60 - DNA, 88 bp, synthetic construct; identical to SEQ ID NO: 45
  misc_feature 1..88: note = Description of Artificial Sequence: Synthetic oligonucleotide
  source 1..88: mol_type = other DNA

SEQ ID NO: 61 - DNA, 137 bp, synthetic construct; identical to SEQ ID NO: 55
  misc_feature 1..137: note = Description of Artificial Sequence: Synthetic polynucleotide
  source 1..137: mol_type = other DNA

SEQ ID NO: 62 - DNA, 137 bp, synthetic construct; identical to SEQ ID NO: 53
  misc_feature 1..137: note = Description of Artificial Sequence: Synthetic polynucleotide
  source 1..137: mol_type = other DNA

SEQ ID NO: 63 - DNA, 137 bp, synthetic construct; identical to SEQ ID NO: 53
  misc_feature 1..137: note = Description of Artificial Sequence: Synthetic polynucleotide
  source 1..137: mol_type = other DNA

SEQ ID NO: 64 - DNA, 136 bp, synthetic construct
  misc_feature 1..136: note = Description of Artificial Sequence: Synthetic polynucleotide
  source 1..136: mol_type = other DNA
ttagcggccgcgaattcgcccttcctgtgacctgtggagacacggccagatctgccctccagcctgatcttttggccagaaggagattaaaaagatgcccctcaagatggcaagggcgaattcgtttaaacctgca

SEQ ID NO: 65 - DNA, 133 bp, synthetic construct; identical to SEQ ID NO: 48
  misc_feature 1..133: note = Description of Artificial Sequence: Synthetic polynucleotide
  source 1..133: mol_type = other DNA

SEQ ID NO: 66 - DNA, 133 bp, synthetic construct; identical to SEQ ID NO: 48
  misc_feature 1..133: note = Description of Artificial Sequence: Synthetic polynucleotide
  source 1..133: mol_type = other DNA

SEQ ID NO: 67 - DNA, 65 bp, synthetic construct
  misc_feature 1..65: note = Description of Artificial Sequence: Synthetic oligonucleotide
  source 1..65: mol_type = other DNA
ttggccagaaggagattaaaaagatgcccctcaagatggcaagggcgaattcgtttaaacctgca

SEQ ID NO: 68 - DNA, 135 bp, synthetic construct
  misc_feature 1..135: note = Description of Artificial Sequence: Synthetic polynucleotide
  source 1..135: mol_type = other DNA
tttagcggccgcgaattcgcccttcctgtgacctgtggagacacggccagatctgccctccagcctgatcttttggccagaaggagattaaaaagatgcccctcaagatggcaagggcgaattcgtttaaacctg

SEQ ID NO: 69 - DNA, 135 bp, synthetic construct; identical to SEQ ID NO: 68
  misc_feature 1..135: note = Description of Artificial Sequence: Synthetic polynucleotide
  source 1..135: mol_type = other DNA

SEQ ID NO: 70 - DNA, 135 bp, synthetic construct; identical to SEQ ID NO: 68
  misc_feature 1..135: note = Description of Artificial Sequence: Synthetic polynucleotide
  source 1..135: mol_type = other DNA

SEQ ID NO: 71 - DNA, 112 bp, synthetic construct
  misc_feature 1..112: note = Description of Artificial Sequence: Synthetic polynucleotide
  source 1..112: mol_type = other DNA
tttagcggccgcgaattcgcccttcctgtgacctgtggagacacggccagatctgccctccagcctgatcttttggccagaaggagattaaaaagatgcccctcaagatggc

SEQ ID NO: 72 - DNA, 48 bp, synthetic construct
  misc_feature 1..48: note = Description of Artificial Sequence: Synthetic oligonucleotide
  source 1..48: mol_type = other DNA
tttagcggccgcgaattcgcccttcctgtgacctgtggagacacggcc

SEQ ID NO: 73 - DNA, 135 bp, synthetic construct; identical to SEQ ID NO: 68
  misc_feature 1..135: note = Description of Artificial Sequence: Synthetic polynucleotide
  source 1..135: mol_type = other DNA

SEQ ID NO: 74 - DNA, 88 bp, synthetic construct; identical to SEQ ID NO: 45
  misc_feature 1..88: note = Description of Artificial Sequence: Synthetic oligonucleotide
  source 1..88: mol_type = other DNA

SEQ ID NO: 75 - DNA, 135 bp, synthetic construct; identical to SEQ ID NO: 68
  misc_feature 1..135: note = Description of Artificial Sequence: Synthetic polynucleotide
  source 1..135: mol_type = other DNA

SEQ ID NO: 76 - DNA, 135 bp, synthetic construct; identical to SEQ ID NO: 68
  misc_feature 1..135: note = Description of Artificial Sequence: Synthetic polynucleotide
  source 1..135: mol_type = other DNA

SEQ ID NO: 77 - DNA, 135 bp, synthetic construct; identical to SEQ ID NO: 68
  misc_feature 1..135: note = Description of Artificial Sequence: Synthetic polynucleotide
  source 1..135: mol_type = other DNA

SEQ ID NO: 78 - DNA, 133 bp, synthetic construct
  misc_feature 1..133: note = Description of Artificial Sequence: Synthetic polynucleotide
  source 1..133: mol_type = other DNA
tagcggccgcgaattcgcccttcctgtgacctgtggagacacggccagatctgccctccagcctgatcttttggccagaaggagattaaaaagatgcccctcaagatggcaagggcgaattcgtttaaacctg

SEQ ID NO: 79 - DNA, 66 bp, synthetic construct
  misc_feature 1..66: note = Description of Artificial Sequence: Synthetic oligonucleotide
  source 1..66: mol_type = other DNA
cttttggccagaaggagattaaaaagatgcccctcaagatggcaagggcgaattcgtttaaacctg

SEQ ID NO: 80 - DNA, 61 bp, synthetic construct
  misc_feature 1..61: note = Description of Artificial Sequence: Synthetic oligonucleotide
  source 1..61: mol_type = other DNA
ggccagaaggagattaaaaagatgcccctcaagatggcaagggcgaattcgtttaaacctg

SEQ ID NO: 81 - DNA, 109 bp, synthetic construct
  misc_feature 1..109: note = Description of Artificial Sequence: Synthetic polynucleotide
  source 1..109: mol_type = other DNA
tttagcggccgcgaattcgcccttcctgtgacctgtggagacacggccagatctgccctccagcctgatcttttggccagaaggagattaaaaagatgcccctcaagat

SEQ ID NO: 82 - DNA, 136 bp, synthetic construct
  misc_feature 1..136: note = Description of Artificial Sequence: Synthetic polynucleotide
  source 1..136: mol_type = other DNA
tttagcggccgcgaattcgcccttcctgtgacctgtggagacacggccagatctgccctccagcctgatcttttggccagaaggagattaaaaagatgcccctcaagatggcaagggcgaattcgtttaaacctgc

SEQ ID NO: 83 - DNA, 136 bp, synthetic construct; identical to SEQ ID NO: 82
  misc_feature 1..136: note = Description of Artificial Sequence: Synthetic polynucleotide
  source 1..136: mol_type = other DNA

SEQ ID NO: 84 - DNA, 136 bp, synthetic construct; identical to SEQ ID NO: 82
  misc_feature 1..136: note = Description of Artificial Sequence: Synthetic polynucleotide
  source 1..136: mol_type = other DNA

SEQ ID NO: 85 - DNA, 136 bp, synthetic construct; identical to SEQ ID NO: 82
  misc_feature 1..136: note = Description of Artificial Sequence: Synthetic polynucleotide
  source 1..136: mol_type = other DNA

SEQ ID NO: 86 - DNA, 109 bp, synthetic construct; identical to SEQ ID NO: 81
  misc_feature 1..109: note = Description of Artificial Sequence: Synthetic polynucleotide
  source 1..109: mol_type = other DNA

SEQ ID NO: 87 - DNA, 136 bp, synthetic construct; identical to SEQ ID NO: 82
  misc_feature 1..136: note = Description of Artificial Sequence: Synthetic polynucleotide
  source 1..136: mol_type = other DNA

SEQ ID NO: 88 - DNA, 88 bp, synthetic construct; identical to SEQ ID NO: 45
  misc_feature 1..88: note = Description of Artificial Sequence: Synthetic oligonucleotide
  source 1..88: mol_type = other DNA

SEQ ID NO: 89 - DNA, 136 bp, synthetic construct; identical to SEQ ID NO: 82
  misc_feature 1..136: note = Description of Artificial Sequence: Synthetic polynucleotide
  source 1..136: mol_type = other DNA

SEQ ID NO: 90 - DNA, 136 bp, synthetic construct; identical to SEQ ID NO: 82
  misc_feature 1..136: note = Description of Artificial Sequence: Synthetic polynucleotide
  source 1..136: mol_type = other DNA

SEQ ID NO: 91 - DNA, 136 bp, synthetic construct; identical to SEQ ID NO: 82
  misc_feature 1..136: note = Description of Artificial Sequence: Synthetic polynucleotide
  source 1..136: mol_type = other DNA

SEQ ID NO: 92 - DNA, 134 bp, synthetic construct
  misc_feature 1..134: note = Description of Artificial Sequence: Synthetic polynucleotide
  source 1..134: mol_type = other DNA
tagcggccgcgaattcgcccttcctgtgacctgtggagacacggccagatctgccctccagcctgatcttttggccagaaggagattaaaaagatgcccctcaagatggcaagggcgaattcgtttaaacctgc

SEQ ID NO: 93 - DNA, 133 bp, synthetic construct
  misc_feature 1..133: note = Description of Artificial Sequence: Synthetic polynucleotide
  source 1..133: mol_type = other DNA
agcggccgcgaattcgcccttcctgtgacctgtggagacacggccagatctgccctccagcctgatcttttggccagaaggagattaaaaagatgcccctcaagatggcaagggcgaattcgtttaaacctgc

SEQ ID NO: 94 - DNA, 84 bp, synthetic construct
  misc_feature 1..84: note = Description of Artificial Sequence: Synthetic oligonucleotide
  source 1..84: mol_type = other DNA
ctgccctccagcctgatcttttggccagaaggagattaaaaagatgcccctcaagatggcaagggcgaattcgtttaaacctgc

SEQ ID NO: 95 - DNA, 67 bp, synthetic construct
  misc_feature 1..67: note = Description of Artificial Sequence: Synthetic oligonucleotide
  source 1..67: mol_type = other DNA
cttttggccagaaggagattaaaaagatgcccctcaagatggcaagggcgaattcgtttaaacctgc